(19) United States
(12) Patent Application Publication
(10) Pub. No.: US 2025/0347760 A1
(43) Pub. Date: Nov. 13, 2025

(54) MAGNETIC RESONANCE SYSTEM TRANSPORT APPARATUS AND MAGNETIC RESONANCE SYSTEM TRANSPORT METHOD

(71) Applicant: GE Precision Healthcare LLC, Waukesha, WI (US)

(72) Inventors: Xiaowu Du, Beijing (CN); Hailiang Liu, Beijing (CN); Zhijun Zhang, Beijing (CN); Huaiyu Dong, Beijing (CN); Zeyu Zhong, Beijing (CN)

(21) Appl. No.: 19/204,061

(22) Filed: May 9, 2025

(30) Foreign Application Priority Data
May 11, 2024 (CN) 202410584682.9

(51) Int. Cl. G01R 33/38; G01R 33/3815

(52) U.S. Cl. CPC G01R 33/3804; G01R 33/3815

(57) ABSTRACT

A magnetic resonance system transport apparatus and a magnetic resonance system transport method are provided. The apparatus includes an integrated container body, in which a compressor and a cooling system are provided. The compressor is connected via a cooling pipe to a cold head of a magnetic resonance system arranged outside the integrated container body, so as to supply cooling capacity to the cold head. The cooling system is used to perform heat exchange with the compressor in the integrated container body, so as to perform refrigeration on the compressor.

### CROSS REFERENCE TO RELATED APPLICATIONS

The present application claims priority and benefit of Chinese Patent Application No. 202410584682.9 filed on May 11, 2024, which is incorporated herein by reference in its entirety.

### TECHNICAL FIELD

The present invention relates to medical imaging systems, and particularly, to a magnetic resonance system transport apparatus and a magnetic resonance system transport method.

### BACKGROUND

A magnetic resonance system comprises a superconducting magnet, which is brought to a superconducting state in a cryogenic environment so as to maintain a required main magnetic field strength. Helium, which is relatively expensive, is typically used as a refrigerant to provide a cooling environment for the superconducting magnet. Loss of the refrigerant may be caused when the temperature rises, and therefore, the refrigerant needs to be replenished, resulting in a large cost waste. As a result, it is necessary to cool the refrigerant during transport even if coils of the superconducting magnet are not energized, to prevent the refrigerant from being volatilized in an environment in which the temperature is rising. This cooling is typically implemented by means of a compressor (e.g., a helium compressor). During transport, the superconducting magnet and the compressor are typically provided in a container and the compressor is cooled by means of an air conditioning system provided in the container, so as to ensure proper operation of the compressor.

### BRIEF DESCRIPTION

According to a first aspect of the present invention, a magnetic resonance system transport apparatus is provided, comprising an integrated container body, in which a compressor and a cooling system are provided. The compressor is connected via a cooling pipe to a cold head of a magnetic resonance system arranged outside the integrated container body, so as to supply cooling capacity to the cold head. The cooling system is used to perform heat exchange with the compressor in the integrated container body, so as to perform refrigeration on the compressor.

According to a second aspect of the present invention, the cooling system comprises a first water-cooling module and a second water-cooling module, and the first water-cooling module and the second water-cooling module alternately perform heat exchange with the compressor.

According to a third aspect of the present invention, a magnetic resonance system transport method is provided, wherein a magnetic resonance system is transported by using the magnetic resonance system transport apparatus of the second aspect. The method comprises executing at least one of the following steps: periodically switching the first water-cooling module and the second water-cooling module; and the method comprises executing at least one of the following steps: 1) periodically switching the first water-cooling module and the second water-cooling module; and 2) periodically resetting the compressor.

It should be understood that the brief description above is provided to introduce, in a simplified form, concepts that will be further described in the detailed description. The brief description above is not meant to identify key or essential features of the claimed subject matter. The scope is defined uniquely by the claims that follow the detailed description. Furthermore, the claimed subject matter is not limited to implementations that solve any deficiencies raised above or in any section of the present disclosure.

### DRAWINGS

The present invention will be better understood by reading the following description of non-limiting examples with reference to the accompanying drawings, wherein

FIG. 1 illustrates a schematic diagram of a magnetic resonance system according to some embodiments;

FIG. 2 illustrates a schematic diagram of a structure for performing refrigeration on a superconducting magnet of a magnetic resonance system by using a compressor according to some other embodiments;

FIG. 3 illustrates a schematic three-dimensional structural diagram of a magnetic resonance system transport apparatus according to some embodiments of the present application;

FIG. 4 and FIG. 5 respectively illustrate different application scenarios of a magnetic resonance system transport apparatus according to the embodiments of the present application;

FIG. 6 illustrates a schematic diagram of a structure of an integrated container body according to some embodiments of the present application;

FIG. 7 illustrates a schematic diagram of a structure of a first water-cooling module according to an example of the present application;

FIG. 8 illustrates a schematic diagram of a structure of an integrated container body according to some other embodiments of the present application;

FIG. 9 illustrates a schematic diagram of a structure of an integrated container body according to some other embodiments of the present application;

FIG. 10 illustrates a schematic diagram of a structure of an integrated container body according to some other embodiments of the present application;

FIG. 11 illustrates a schematic diagram of a structure of a shock absorbing and damping apparatus according to some embodiments of the present application;

FIG. 12 illustrates a cross-sectional view of the shock absorbing and damping apparatus; and

FIG. 13, FIG. 14 and FIG. 15 illustrate flow charts of a magnetic resonance system transport method according to some embodiments of the present application.

The accompanying drawings illustrate described components of the magnetic resonance system transport apparatus and steps of the magnetic resonance system transport method. Together with the following description, the accompanying drawings illustrate and explain structural principles, methods, and principles described herein. In the accompanying drawings, the thickness and dimensions of the components may be enlarged or otherwise modified for clarity. Well-known structures, materials, or operations are not shown or described in detail to prevent the described components, systems, and methods from being obscured.

### DETAILED DESCRIPTION

Specific implementations of the present invention will be described below. It should be noted that in the specific description of said implementations, for the sake of brevity and conciseness, the present description cannot describe all of the features of the actual implementations in detail. It should be understood that in the actual implementation process of any implementation, just as in the process of any one engineering project or design project, a variety of specific decisions are often made to achieve specific goals of the developer and to meet system-related or business-related constraints, which may also vary from one implementation to another. Furthermore, it should also be understood that although efforts made in such development processes may be complex and tedious, for those of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless otherwise defined, the technical or scientific terms used in the claims and the description should be as they are usually understood by those possessing ordinary skill in the technical field to which they belong. Terms such as “first”, “second”, and similar terms used in the present description and claims do not denote any order, quantity, or importance, but are only intended to distinguish different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections. Furthermore, it should be understood that references to “an example” or “examples” of the present disclosure are not intended to be construed as excluding the existence of additional implementations that also incorporate the referenced features.

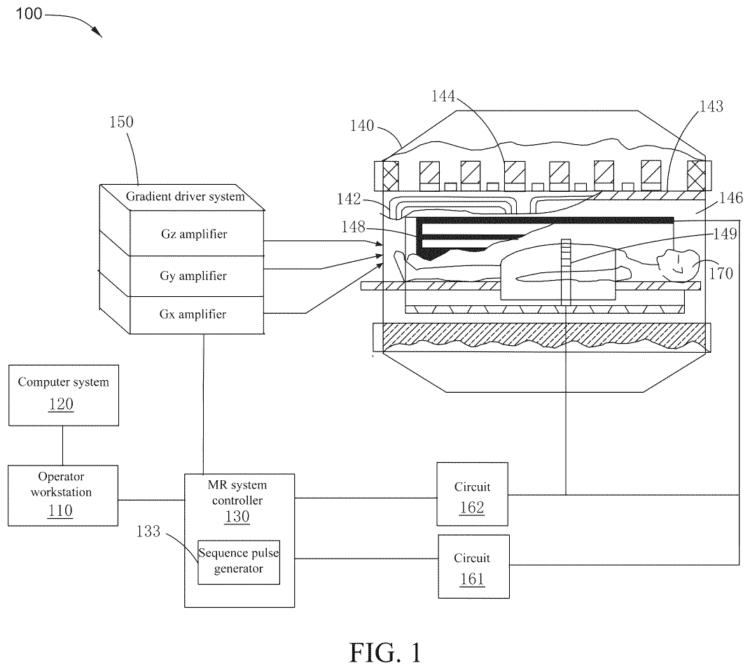

Referring to FIG. 1, a schematic diagram of an exemplary magnetic resonance (M R) system 100 according to some embodiments is illustrated. An operator workstation 110 is used to control operation of the M R system 100. The operator workstation 110 is coupled to and in communication with a computer system 120. The computer system 120 may be used to process and store image data, such as M R signals, generated by the M R system. The computer system 120 may be coupled to and in communication with an M R system controller 130.

The M R system controller 130 may include a sequence pulse generator 133 which is in communication with the operator workstation 110. In some embodiments, at least part of the sequence pulse generator 133 may be integrated into a magnetic resonance assembly 140 of the M R system 100.

The magnetic resonance assembly 140 includes a superconducting magnet 143. The superconducting magnet 143 has a magnet bore, so as to form a cylindrical imaging volume 146, used to accommodate a scanned subject 170 during operation of the M R system. The superconducting magnet has superconducting coils 144, and during operation, the superconducting coils 144 provide a static uniform longitudinal magnetic field Bo throughout the cylindrical imaging volume 146.

The magnetic resonance assembly 140 further includes a radio-frequency coil assembly and a gradient coil assembly 142. The radio-frequency coil assembly may include, for example, a body coil 148 and a surface coil 149, which may be used to send and/or receive a radio-frequency pulse signal. The gradient coil assembly 142 is used to receive a gradient pulse signal.

The M R system controller 130 may receive a command from the operator workstation 110 to indicate an M R scan sequence that is to be executed during an M R scan. The sequence pulse generator 133 of the M R system controller 130 generates, on the basis of the indicated scan sequence, a radio frequency pulse and a gradient pulse.

The radio frequency pulse sent by the sequence pulse generator 133 is processed and amplified via a circuit 161 and is then provided to the body coil 148, and the body coil 148 in turn provides a transverse magnetic field B₁. The transverse magnetic field B₁ is substantially perpendicular to B₀ throughout the entire cylindrical imaging volume 146. The transverse magnetic field B₁ is used to excite stimulated nuclei (or protons) in the body of the scanned subject so as to generate an M R signal.

The gradient pulse sent by the sequence pulse generator 133 is sent to a gradient driver 150. The gradient driver 150 includes Gₓ, G_{y}, and G_{z} amplifiers, etc. Each of the Gₓ, G_{y}, and G_{z} gradient amplifiers is used to excite, on the basis of the gradient pulse, a corresponding gradient coil in the gradient coil assembly 142, so as to generate a gradient magnetic field superimposed on a static magnetic field, and is used to generate a magnetic gradient for performing spatial encoding on an M R signal during an M R scan.

The RF body coil 148 and the RF surface coil 149 may be used to transmit a radio frequency pulse and/or receive an M R signal from the subject when performing magnetic resonance scan on the subject. The M R signal may be sensed and received by the RF body coil 148 or the surface coil 149 and processed by means of a circuit 162 to then form an image data array. A processor (not shown) in the M R system controller 130 is used to generate a reconstructed image according to the image data. In response to the command received from the operator workstation 110, these images may be transmitted to the computer system 120 for further processing and to the operator workstation 110 for presentation via a display (not shown) of the operator workstation 110.

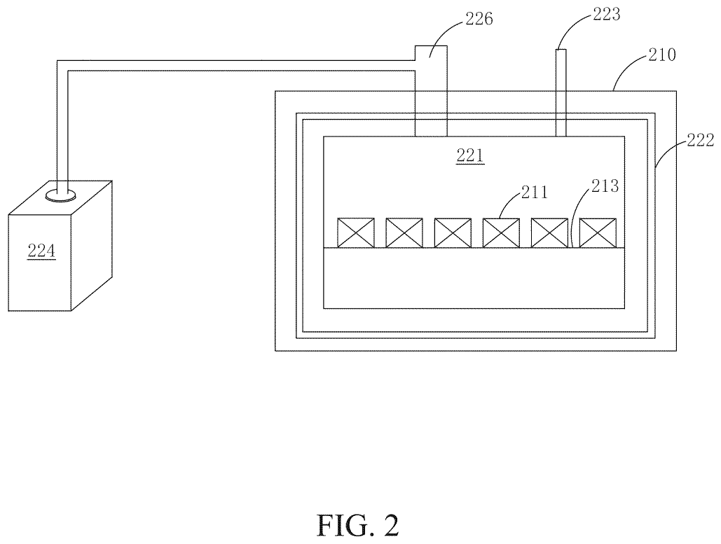

FIG. 2 illustrates a schematic diagram of a structure for performing refrigeration on a superconducting magnet of a magnetic resonance system by using a compressor according to some other embodiments. A magnetic resonance assembly 210 and a compressor 224 are shown. The magnetic resonance assembly 210 may include part or all of the assembly 140 in FIG. 1, for example, including a superconducting magnet 213 and superconducting coils 211. As described above, the superconducting coils 211 are used to generate a main magnetic field, and the superconducting coils 211 need to be cooled to a superconducting state to maintain a required main magnetic field strength. For this purpose, the superconducting coils 211 are immersed in a cryogenic container 221. The cryogenic container 221 is used to contain a cryogenic refrigerant. A part from being immersed in a cryogenic container, the superconducting coils 211 may perform heat exchange with the cryogenic refrigerant in other ways so as to achieve a required temperature. Typically, liquid helium may be used as the cryogenic refrigerant. Specifically, the cryogenic container 221 may surround the superconducting magnet 213 in which the superconducting coils 211 are located. The cryogenic container 221 may be provided in a thermal shield 222. A vacuum insulation region may be arranged between the thermal shield 222 and the cryogenic container 221. The thermal shield 222 and the vacuum insulation region isolate the cryogenic refrigerant from an external heat source, so as to prevent the cryogenic refrigerant from being volatilized. The cryogenic container 221 is provided with a pressure relief valve 223, used to relieve excessive pressure caused by, for example, volatilization of the refrigerant. The cryogenic refrigerant needs to be prevented from being volatilized during operation or transport of the magnetic resonance system. On the one hand, the volatilization of the refrigerant may cause the superconducting magnet to quench and re-excitation may cause a large cost. On the other hand, the cryogenic refrigerant is expensive and its volatilization may cause a large cost waste.

The compressor 224 is used to cool the refrigerant in the cryogenic container 221. Specifically, the compressor 224 provides cooling capacity to a cold head 226 of the magnetic resonance system. The cold head in turn cools the refrigerant in the cryogenic container 221 by performing heat exchange with the cryogenic container 221.

Typically, a refrigerant needs to be filled in the cryogenic container 221 before shipment of the magnetic resonance system, so as to prevent such a complicated operation at an mounting site. Therefore, the refrigerant in the cryogenic container 221 also needs to be kept in a cryogenic state during transport. For this purpose, it is desirable to ensure proper operation of the compressor as much as possible so as to ensure the cooling effect of the refrigerant.

The compressor 224 needs to be refrigerated for proper operation. Therefore, an air conditioner is typically mounted in the container for transport, so as to ensure proper operation of the compressor 224, thereby preventing the refrigerant in the cryogenic container 221 from being volatilized to the outside environment due to a temperature rise.

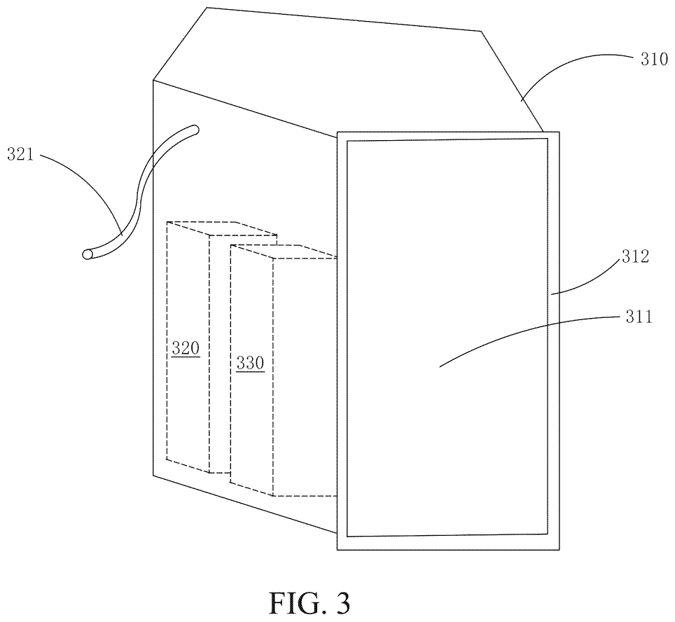

FIG. 3 illustrates a schematic three-dimensional structural diagram of a magnetic resonance system transport apparatus according to some embodiments of the present application. The transport apparatus includes an integrated container body 310. The integrated container body 310 is used to carry a compressor 320 and a cooling system 330. The cooling system 330 cools the compressor in the integrated container body 310, and the integrated container body 310 is spatially separated from a component to be transported (e.g., the superconducting magnet) of the magnetic resonance system, so that the integrated container body 310 and the component carried thereby can serve as standard members to adapt to different transport tools. For example, the integrated container body 310 as a whole can be placed together with the superconducting magnet in an on-board container, or arranged on a transport truck by means of a bracket and located outside the container of the superconducting magnet, or as a whole may be placed in a train carriage, or may serve as part of a marine assembly for combining with a marine dedicated container. After the transport ends, the integrated container body 310 can be removed from the corresponding container or bracket. Therefore, the flexibility is high, the transport efficiency is improved, there is no need to provide different transport solutions to adapt to different transport conditions, the cooling environment conditions of the magnet container can be adapted to, and transport operation errors are reduced due to consistent operations brought about by the integrated container body.

Specifically, the compressor 320 and the cooling system 330 are provided in the integrated container body 310. An example of the compressor 320 is the compressor 224 described above. The transported magnetic resonance system may include components of the magnetic resonance system or variants thereof in any of the embodiments above.

Referring to FIG. 3, the compressor 320 is connected to the cold head of the magnetic resonance system 301 via a refrigerant pipe 321. The refrigerant pipe 321, the magnetic resonance system 301, and the cold head of the magnetic resonance system 301 are all provided outside the integrated container body 310. One end of the refrigerant pipe 321 is in communication with the compressor 320 via an interface (not shown) provided on the integrated container body. Specifically, a gaseous refrigerant (e.g., helium) generated at the cold head due to heat exchange enters the compressor 320 via the refrigerant pipe 321. The compressor 320 compresses the gaseous refrigerant to form a liquid refrigerant. The liquid refrigerant carries cooling capacity and enters the cold head via the refrigerant pipe 321. In some embodiments, the integrated container body 310 has a volume smaller than the container carrying the magnetic resonance system 301 so as to be able to enter and exit from the container.

The cooling system 330 is also provided in the integrated container body 310 and used to perform heat exchange with the compressor 320 in the integrated container body 310, so as to perform refrigeration on the compressor 320. In this way, it is not necessary to cool the compressor 320 using an external cooling device during transport again.

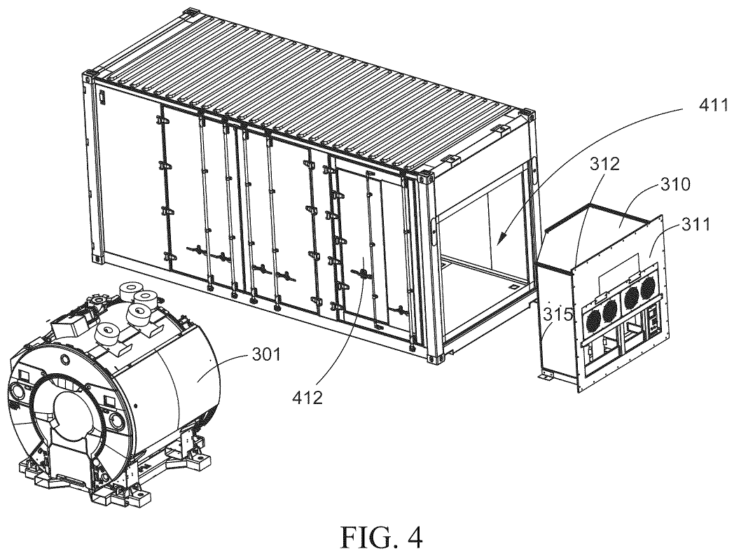

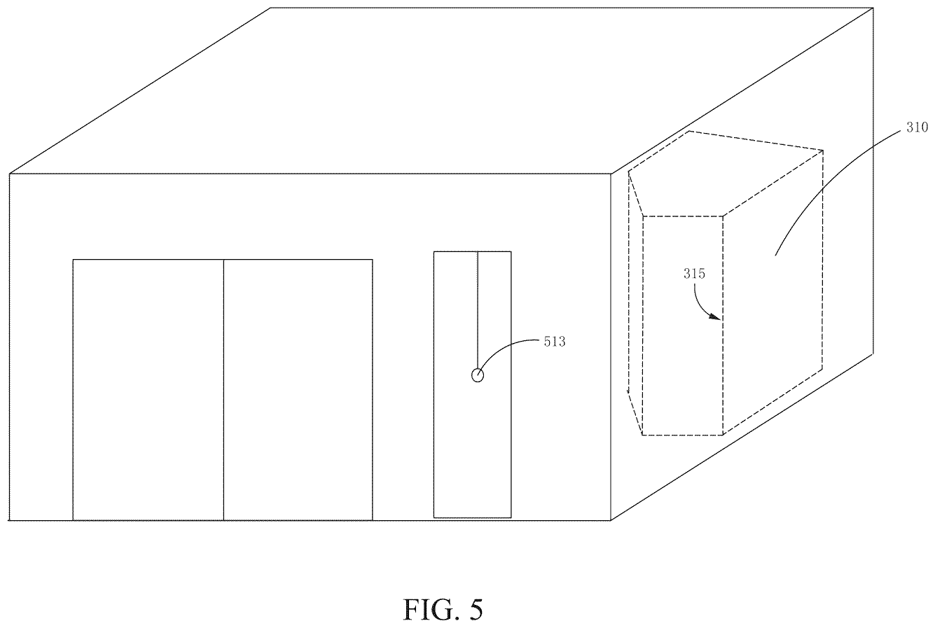

In some embodiments, the transport apparatus further includes a container, used to accommodate the magnetic resonance system 301 and integrated container body 310 to be transported. The integrated container body 310 can be removed from the container. FIG. 4 and FIG. 5 respectively illustrate different application scenarios of a magnetic resonance system transport apparatus according to the embodiments of the present application. As shown in FIG. 4, a container 410 is provided with an opening 411, via which at least part of the integrated container body 310 is capable of being provided in the container 410. The integrated container body 310 is in communication with the outside of the container 410 via the opening. For example, the integrated container body 310 may entirely enter the container via the opening 411 (in other embodiments, alternatively via a door of the container 410), or part of the volume of the integrated container body 310 is located in the container 410 via the opening 411. At least one side of the integrated container body 310 may be in communication with the opening 411. In other words, the at least one side is exposed from the opening 411, allowing an operator to monitor or operate a device on the integrated container body 310.

In an embodiment, the integrated container body 310 includes an outer side portion 311. The outer side portion 311 is in communication with the outside of the container 410 via the opening 411. The outer side portion 311 may have a shape and a size matching the opening 411. In some embodiments, an edge portion of the outer side portion 311 is provided with a flange 312, used to connect a wall of the opening 411. The flange 312 may be provided at least partially surrounding the outer side portion 311.

The container 410 and the integrated container body 310 described above may be configured in a kit for marine transport. For example, the container 410 is placed on a ship, and the integrated container body 310 is placed in the container 410 and is visually integrated with the container 410. For example, the outer side portion 311 of the integrated container body 310 is substantially in the same plane as the wall on which the opening 411 is located, or combined to form an integrated pattern or shape.

The container 410 described above may further be provided with a door 412 for an operator to pass through. The door 412 and the opening 411 may be located on the same side or different sides of the container 410. In some embodiments, the door 412 is arranged between a region in which the magnetic resonance system 301 is placed and a region in which the integrated container body 310 is placed, so as to facilitate manipulation of any one of the magnetic resonance system 301 and the integrated container body 310.

As shown in FIG. 5, the integrated container body 310 may be provided as an independent device in a closed container 510. The closed container 510 does not need to be provided with an opening for placing the integrated container body in communication with the outside. Therefore, the container 510 may be any existing container, for example, an on-board container, a train carriage, or another cold chain transport container.

During transport, the integrated container body as a whole can be dismantled and reloaded when the transport tool needs to be replaced.

In some embodiments of the present application, the integrated container body 310 has a non-rectangular polygonal cross-section. For example, as shown in FIG. 4, the cross-section of the integrated container body 310 is an irregular pentagon. A volume formed by a relatively narrow side and the other two sides adjacent thereto is provided in the container 410 (e.g., the inner side of the opening 411), and a relatively wide side opposite to the relatively narrow side is in communication with the outside of the container 410 via the opening 411. By means of such a design, in and out of the integrated container body 310 via the opening 411 is facilitated, and a space is formed near the portion of the integrated container body 310 close to the door 412, so that an operator can perform manipulation and maintenance on the integrated container body 310 behind the door 412.

In addition, the containers 410 and 510 are provided with emergency handles (not shown in FIG. 4) 513, respectively, for opening the doors on the containers in case of emergency.

In some embodiments, the outer side portion 311 of the integrated container body 310 is provided with an operation window or operation door, to facilitate monitoring and operation of a device. For example, an operator may observe or operate at least part of a water-cooling system, at least part of a control and communication system, and/or at least part of a power supply module to be described below, via the operation window or the operation door of the outer side portion 311, i.e., at least part of these systems can be presented to the operator by means of the outer side portion 311.

As shown in FIG. 4 and FIG. 5, an insulation layer 315 may be provided on the inner side of the integrated container body 310, to insulate heat exchange with the outside of the integrated container body 310. Further, a seal member (not shown) is arranged between adjacent panels forming the integrated container body 310.

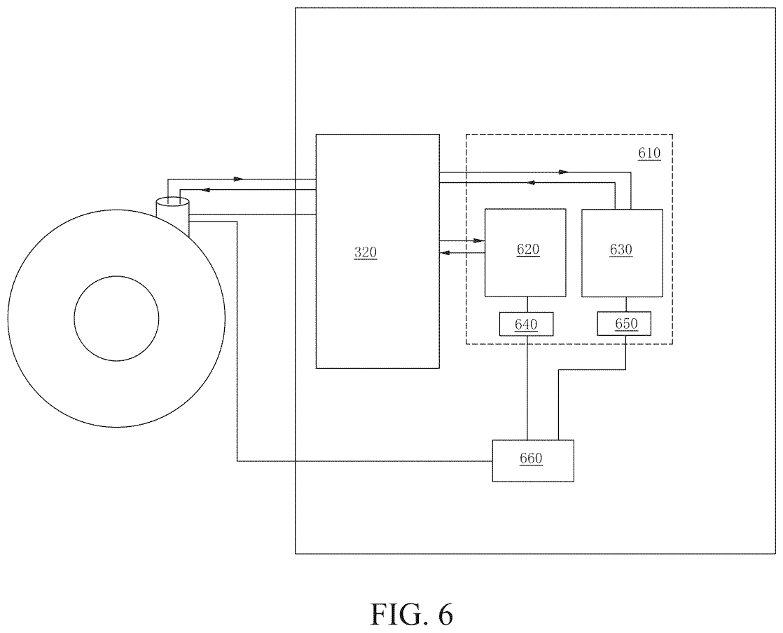

FIG. 6 illustrates a schematic diagram of a structure of an integrated container body 310 according to some embodiments of the present application. Here, a cooling system 330 provided in the integrated container body 310 includes a water-cooling system 610. Refrigeration is performed on the compressor 320 by means of the water-cooling system 610 closer to the compressor 320, so that refrigeration efficiency can be improved and temperature control can be performed more accurately, allowing the compressor to operate in a stable temperature environment.

In some embodiments, the water-cooling system 610 includes a water flow circuit. The water flow circuit performs heat exchange with the compressor 320, to transfer cooling capacity to the compressor.

Continuously referring to FIG. 6, in an embodiment of the present application, the water-cooling system 610 may include a first water-cooling module 620 and a second water-cooling module 630. The first water-cooling module 620 and the second water-cooling module 630 alternately perform heat exchange with the compressor 320. In this way, the reliability of the refrigeration of the compressor is ensured, the volatilization of a superconducting cooling gas due to abnormal operation of the compressor is prevented, and the power consumption is reduced, so that the power supply module provided on a conventional cold chain transport device can satisfy a power requirement of the magnetic resonance transport. However, in some special cases, for example, when the compressor requires more cooling capacity, both the first water-cooling module 620 and the second water-cooling module 630 may be controlled to be turned on to transfer cooling capacity to the compressor simultaneously.

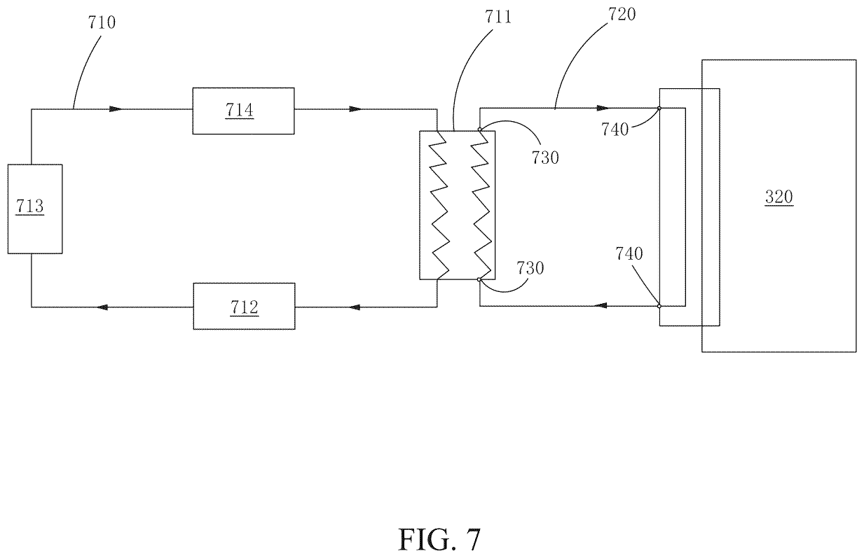

Referring to FIG. 7, a schematic diagram of a structure of the first water-cooling module 610 according to an example of the present application is illustrated. The first water-cooling module 610 includes a first cooling unit 710 and a first water-cooling circuit 720. The first cooling unit 710 is used to circulate a coolant, for example, a Freon fluid. The first cooling unit 710 includes an evaporator 711, a compressor 712, a condenser 713, and an expansion valve 714. The Freon fluid flows through the compressor 712 to be compressed into a high-pressure gas thereby. The high-pressure gas is passed to the condenser 713 to be condensed. The condensed Freon fluid is passed to the expansion valve 714 for pressure reduction and temperature reduction. The Freon fluid undergone the temperature reduction and the pressure reduction is passed to the evaporator 711 to be evaporated into a gas in the evaporator 711. Heat is absorbed in the evaporation process, and the evaporated gas is passed to the compressor 712 to be compressed. The process is circulated.

The first water-cooling circuit 720 is used to circulate cooling water, and devices such as a water tank, a pump, etc., may be provided in the first water-cooling circuit 720. The first water-cooling circuit 720 performs heat exchange with the evaporator 711, so as to absorb cooling capacity generated during the evaporation process of a coolant. The cold water carrying the cooling capacity further performs heat exchange with the compressor 320, so as to perform refrigeration on the compressor 320. The cold water increases in temperature after performing heat exchange with the compressor 320 and continues to circulate in the circuit to again perform heat exchange with the evaporator 711. The process is circulated.

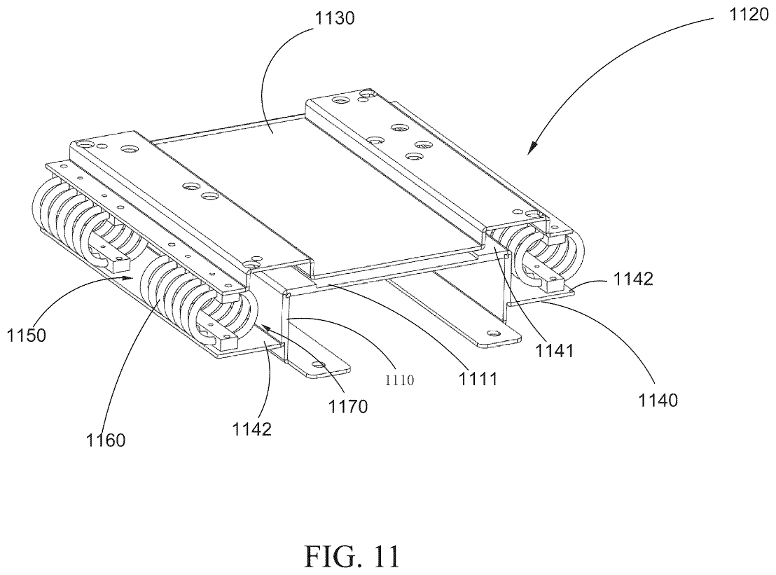

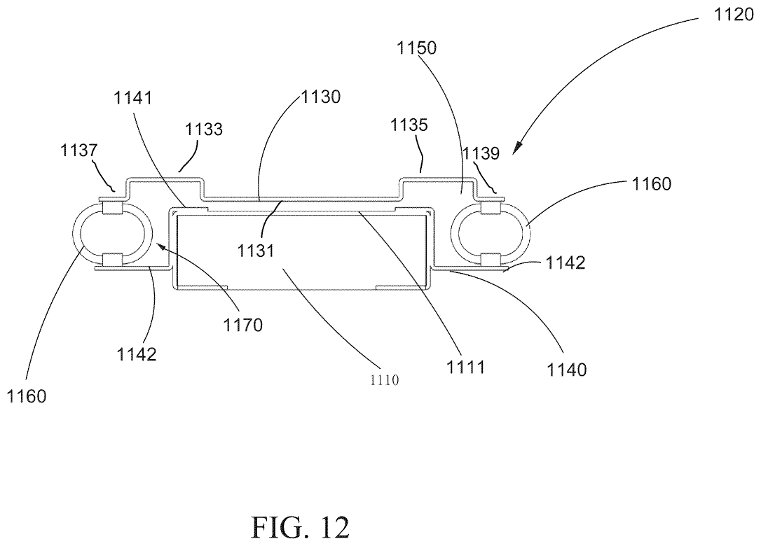

Temperature sensors 730 are provided at a water inlet and a water outlet of the first cooling unit 710, respectively, for monitoring a water inlet temperature and a water outlet temperature of the cooling unit 710, and sending the measured temperatures to a control module, for example, a central controller 660 to be described below, so that the central controller 660 further determines an operating state of the compressor 320 according to received temperature information and performs a corresponding operation on the compressor. In addition, pressure sensors 740 may also be provided at a water inlet and a water outlet of the compressor 320 to measure air pressure. The central controller 660 may determine an operating state of the compressor 320 in combination with the temperature information and air pressure information. These will be described in detail below with reference to FIG. 11.

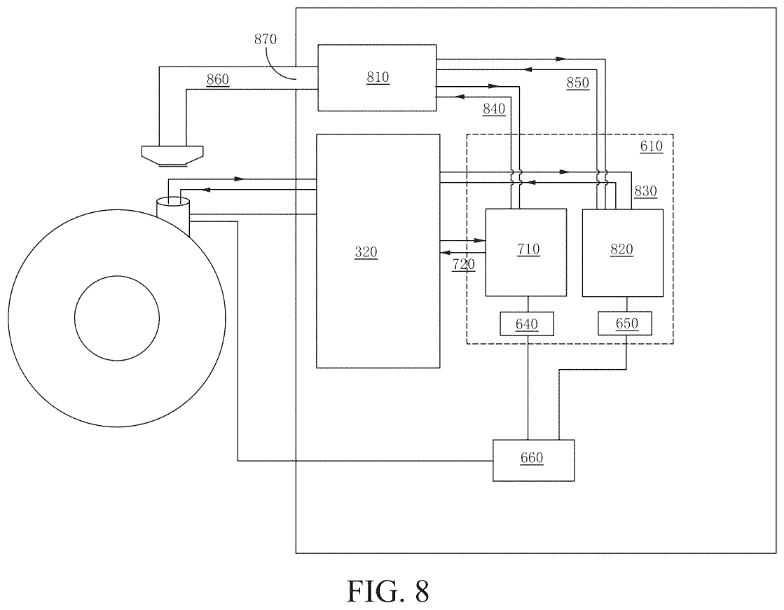

Only one example of the first water-cooling module 610 is shown above, and the first water-cooling module 610 may have other water-cooling principles or structures. The second water-cooling module 630 may have the same structure as the first water-cooling module 620 so as to have a uniform cooling mode and cooling effect for the compressor 320. Specifically, the second water-cooling module 630 may include a second cooling unit 820 and a second water-cooling circuit 830 (as shown in FIG. 8), or the second water-cooling module 630 may include a second cooling unit 820 and the first water-cooling circuit 720, that is, sharing the water-cooling circuit with the first water-cooling module 610, so as to save space and reduce cost.

Continuously referring to FIG. 6, the water-cooling system 610 further includes a first control module 640 and a second control module 650, which are used to communicate with the first water-cooling module 620 and the second water-cooling module 630, respectively, so as to control operating states of the first water-cooling module 620 and the second water-cooling module 630. Specifically, the first control module 640 and the second control module 650 are used to communicate with the central controller 660, so as to control the corresponding first water-cooling module 620 and second water-cooling module 630 on the basis of a control instruction of the central controller 660. For example, the central controller 660 may send a start-up instruction to the first water-cooling module 620 or the second water-cooling module 630 according to a set period of time for operation, so as to instruct the corresponding water-cooling module to enter an operating state. For example, the central controller 660 may shut down a currently operating water-cooling module every 12 hours and start a currently resting water-cooling module simultaneously, so as to implement alternate operation of the water-cooling modules. The central controller 660 may further adjust a period of time during which the first water-cooling module 620 and the second water-cooling module 630 are operated individually or simultaneously according to various feedback information. For example, even if the currently operating first water-cooling module 620 does not reach a preset period of time for operation, when the central controller 660 detects, according to the feedback information, that the first water-cooling module 620 is abnormal, the second water-cooling module 630 starts to be operated instead. In some embodiments, the first control module 640 and the second control module 650 may be programmable logic controllers (PLCs), and the central controller 660 may be any of a computer system, an embedded system, an industrial control system, etc.

Referring to FIG. 8, a schematic diagram of a structure of an integrated container body 310 according to some other embodiments of the present application is illustrated. An air-cooling module 810 is further provided in the integrated container body 310, the air-cooling module 810 performs heat exchange with the water-cooling system 610 to cool the cold head of the magnetic resonance system. Specifically, the air-cooling module 810 may include a fan, which draws air via the interior space of the integrated container body 310 or from the environment outside the integrated container body 310 via the interior space. The air is cooled after heat exchange with the water-cooling system 610. The cooled air is then delivered to the cold head, so as to perform refrigeration on the cold head. By performing air-cooling on the cold head, the power requirements of the compressor 320 are reduced, which in turn reduces the power requirements for performing refrigeration on the compressor 320.

Similar to performing refrigeration on the compressor 320, the first water-cooling module 620 and the second water-cooling module 630 in the water-cooling system 610 may alternately perform heat exchange with the air-cooling module 810. For example, the first water-cooling module 620 may include a third water-cooling circuit 840, the second water-cooling module 630 may include a fourth water-cooling circuit 850, and the third and fourth water-cooling circuits may share a same circuit. In addition, similar to the heat exchange principle of the first water-cooling circuit 720 and the second water-cooling circuit 830 described above, water carrying cooling capacity in the third water-cooling circuit 840 and the fourth water-cooling circuit 850 is used to perform heat exchange with the air-cooling module 810, so that the air-cooling module 810 generates cold air (the cooling air).

In some embodiments, when either of the first water-cooling module 620 and the second water-cooling module 630 is operated, cooling capacity may be provided to both the compressor 320 and the air-cooling module 810, or the heat exchange circuit (e.g., the third water-cooling circuit 840 or the fourth water-cooling circuit 850) of the air-cooling module may be closed, so that cooling capacity is provided to the compressor 320 only.

The air-cooling module 810 delivers the cooling air to the cold head via an air duct 860 that extends outside the integrated container body 310. Specifically, a connecting port 870 is provided on a side wall of the integrated container body 310, one end of the air duct 860 may be in communication with an air outlet of the air-cooling module 810 via the connecting port 870, the air duct 860 is provided outside the integrated container body 310, and the other end thereof can be extended to the cold head of the magnetic resonance system.

Further, the air-cooling module 810 further outputs the cooling air to the compressor within the integrated container body 310 via the heat exchange with the water-cooling system 610 described above, which also reduces the power requirements of the water-cooling unit for performing refrigeration on the compressor.

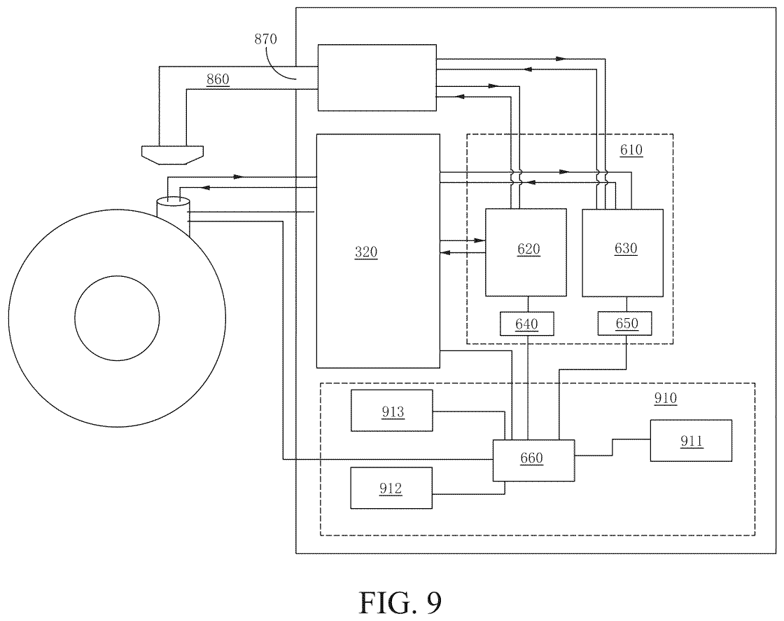

Referring to FIG. 9, a schematic diagram of a structure of a magnetic resonance system transport apparatus according to some other embodiments of the present application is illustrated. The system further includes a control and communication system 910, which includes the central controller 660 described above. As described above, the central controller 660 is communicatively coupled to the first water-cooling module 620 and the second water-cooling module 630 (e.g., directly or by means of the first control module 640 and the second control module 650, respectively), so as to control the first water-cooling module 620 and the second water-cooling module 630 to alternately perform heat exchange with the compressor 320.

The control and communication system 910 may further include at least one of a magnet monitoring module 911, a human-machine interaction module 912, and a remote communication module 913.

The magnet monitoring module 911 is used to receive a main magnet parameter from the magnetic resonance system and send the received main magnet parameter to the central controller 660. The central controller 660 can in turn perform a corresponding operation on the basis of a current state of the main magnet (the superconducting magnet). For example, when the main magnet parameter is abnormal, the central processor may send an instruction so as to adjust a refrigeration state of the compressor, report information via the remote communication module 913, and control the compressor 320 to restart, etc.

The human-machine interaction module 912 is controlled by the central controller so as to display interaction information. For example, feedback data from different monitoring modules or sensors may be displayed for observation by an operator, who may also send an operating instruction to the central controller 660 via the human-machine interaction module 912.

The remote communication module 913 is used to provide a wireless connection between the central controller 660 and a remote device. The remote device may provide a remote control platform that performs interaction with the central controller 660 via any of a gateway, a router, a wireless network, a network cloud, and a satellite network, for example, obtaining feedback data sent via the central controller 660 or remotely sending an operating instruction to the central controller 660.

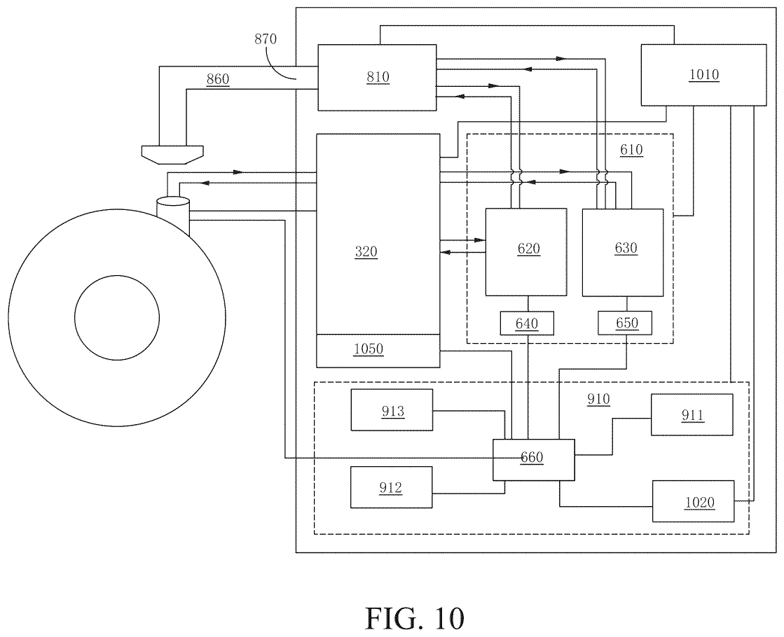

Referring to FIG. 10, a schematic diagram of a structure of the integrated container body 310 according to some other embodiments of the present application is illustrated. A power supply module 1010 is further provided in the integrated container body 310. The power supply module 1010 is used to receive an external power supply and distribute power to at least one of the compressor 320, the water-cooling system 610, the air-cooling module 810, and the control and communication system 910. Specifically, the side wall of the integrated container body 310 is provided with a power supply adapter interface (not shown) for connecting to an external power supply. The external power supply may be a power supply equipped on a transport tool. The power supply module 1010 may include a power adapter unit, used to adjust the power, frequency, phase sequence, etc. of input power of the external power supply, so as to adapt to the corresponding requirements of the compressor 320.

The control and communication system 910 may further include a power supply detection module 1020, used to perform power detection on the power supply module 1010 and upload a power detection result to the central controller 660. The central controller 660 can in turn perform corresponding control on the basis of a power output signal of the power supply module 1010, for example, adjusting power distribution, or controlling on and off of the modules or systems that receive the power distribution.

In the embodiments of the present application, the compressor 320, the water-cooling system 610, the air-cooling module 810, the control and communication system 910, and the power supply module 1010 are respectively provided in a plurality of divided spaces of the integrated container body 310, and these spaces may be divided by means of, for example, a distance or a partition plate. These spaces may be divided in a horizontal direction or a vertical direction so as to have an optimal space distribution. In addition, a plurality of operation windows or operation doors may be provided on a side plate of the integrated container body 310, so as to place these divided spaces in communication separately, facilitating the operation of the systems or the modules therein.

As shown in FIG. 10, the embodiments of the present application may further include a shock absorbing and damping apparatus 1050. The shock absorbing and damping apparatus is provided in the integrated container body 310 so as to carry a component to be damped. The shock absorbing and damping apparatus 1050 is provided at the bottom of the compressor 320 so as to damp the compressor 320 and protect the compressor from an external force. FIG. 11 and FIG. 12 respectively illustrate a three-dimensional schematic diagram and a cross-sectional view of the shock absorbing and damping apparatus 1050 according to the embodiments of the present application.

The shock absorbing and damping apparatus 1050 includes a base support 1110 and a damping part 1120. The base support 1110 may be fastened to the ground or to a supporting surface of the container body. The damping part 1120 includes an upper portion 1130 and a lower portion 1140. The upper portion may be used to carry the compressor 320. The upper portion 1130 forms a unitary body with a central recessed section 1131 defined between two laterally spaced raised portions 1133, 1135. Each raised portion includes a generally vertical wall extending upward from the base (e.g., 1137, 1139) and terminating in a flat or contoured top surface. The central recessed section is bounded laterally by the inner vertical walls of the raised portions, thereby forming a channel or groove. The damping part 1120 further includes a surface 1141 fitting with an upper surface 1111 of the base support 1110 and two lateral extension portions 1142 below the surface 1141. The two lateral extension portions 1142 are respectively arranged in a floating mode on two opposite sides of the base support 1110. A space 1150 is formed between the upper portion 1130 and the lower portion 1140 of the damping part 1120. In addition, steel wire rope dampers 1160 are respectively mounted between the upper portion 1130 of the damping part and the two lateral extension portions 1142. Therefore, the external force in the vertical direction is buffered by using the damping part arranged in a floating mode, the space 1150 between the upper and lower portions of the damping part, and the steel wire rope dampers provided in the space.

Further, a damping space 1170 is formed in a horizontal direction between each of the steel wire rope dampers 1160 and the lower portion 1140 of the damping part 1120, so as to buffer the external force in the horizontal direction. In this way, when the compressor 320 swings in any direction due to an external force generated during transport, the shock absorbing and damping apparatus 1010 gradually converges the swing amplitude from different directions, thereby implementing better buffering, damping, and shock absorbing functions, so as to ensure the stability and reliability of the operation of the compressor.

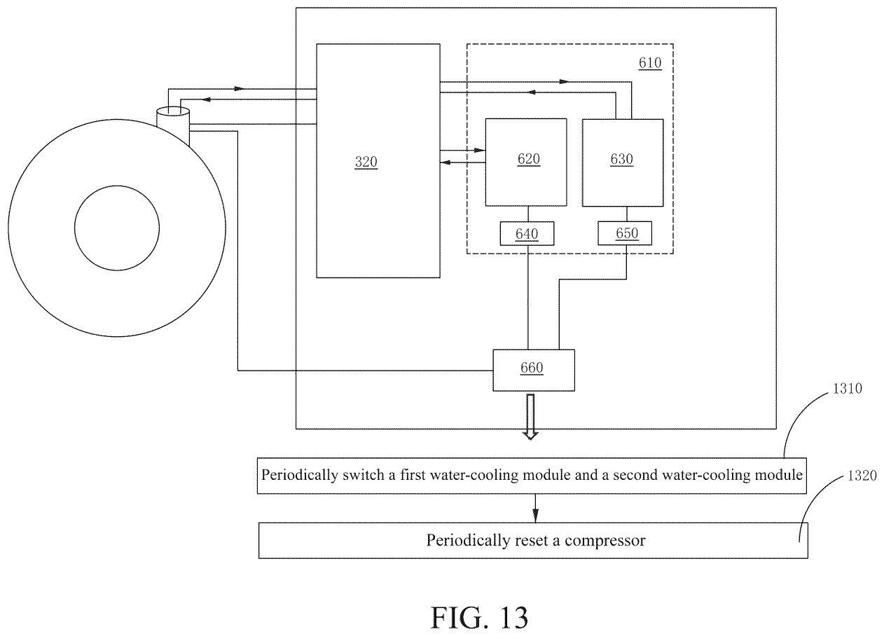

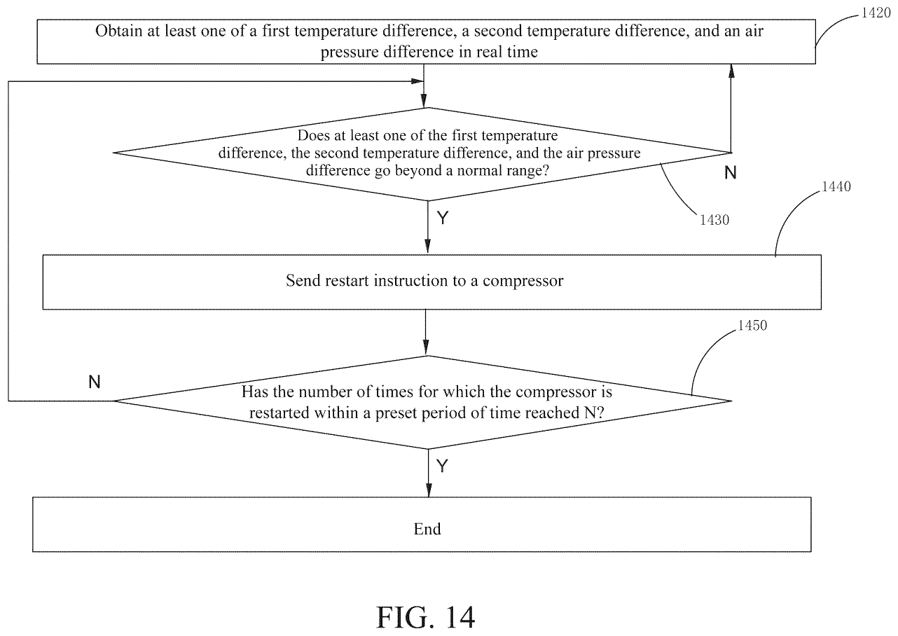

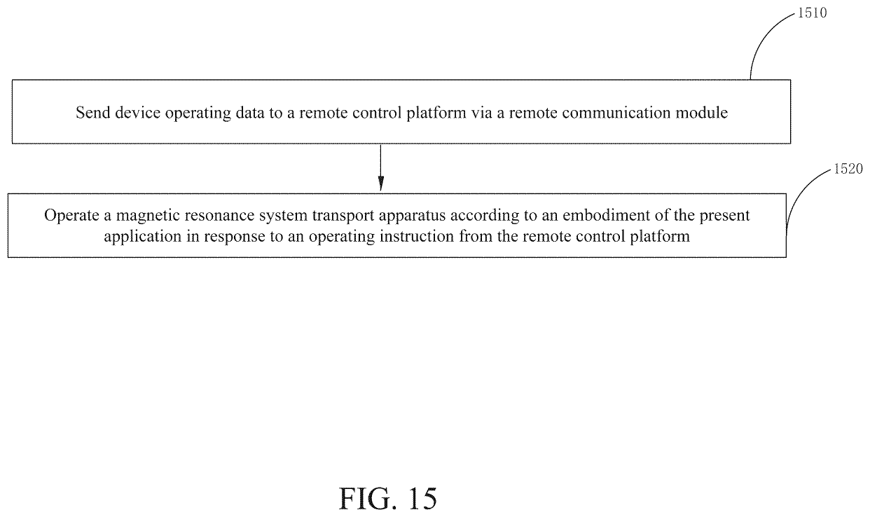

The embodiments of the present invention further provide a magnetic resonance transport method, in which a magnetic resonance system is transported by using the magnetic resonance transport apparatus shown in FIG. 6. FIG. 13 illustrates a flow chart of the magnetic resonance transport method according to some embodiments of the present application. The method includes at least one of step 1310 and step 1320: Step 1310: periodically switching a first water-cooling module 620 and a second water-cooling module 630. Specifically, the central controller 660 may periodically switch the first water-cooling module 620 and the second water-cooling module 630 on the basis of a preset first cycle. For example, when the first cycle is reached, a start-up signal may be sent to one of the water-cooling modules (which is in a non-operating state before a second cycle is reached), and a shut-down signal may be sent to the other water-cooling module (which is in an operating state before the second cycle is reached). In an example, the first cycle is 12 hours. Step 1320: periodically resetting the compressor 320. For example, the central controller 660 may periodically reset the compressor 320 on the basis of the preset second cycle. The resetting may be executed by re-energizing, which allows the compressor to periodically clear stored error information and ignore an anomaly protection mechanism of the compressor, so that the compressor is operated properly to the maximum extent so as to protect the magnet from damage. The second cycle may be set according to a logistic arrival time and a loss of liquid helium during resetting. In an example of the present application, the second cycle may be set to 12 hours or 24 hours.

FIG. 14 illustrates a flow chart of a magnetic resonance transport method according to some other embodiments of the present application. The structures of the first water-cooling module and the second water-cooling module are shown in FIG. 7. Specifically, the first water-cooling module 620 includes a first cooling unit 710 and a first water-cooling circuit 720 that exchanges heat with the first cooling unit 710. The second water-cooling module 630 includes a second cooling unit 820 and a second water-cooling circuit 830 that exchanges heat with the second cooling unit 820. The method includes the following steps:

Step 1420: obtaining at least one of a first temperature difference, a second temperature difference, and an air pressure difference in real time (dynamically), wherein the first temperature difference is a temperature difference between a water outlet temperature and a water inlet temperature of the first cooling unit or the second cooling unit currently in an operating state, the second temperature difference is a temperature difference between a water inlet temperature and a water outlet temperature of the compressor 320, and the air pressure difference is an air pressure difference between an air-inlet air pressure and a water-outlet air pressure of the compressor 320.

For example, the central controller 660 may obtain temperature and air pressure data from corresponding temperature sensors or air pressure sensors in real time and calculate a corresponding temperature difference and air pressure.

Step 1430: executing first determination, wherein it is determined whether at least one of the first temperature difference, the second temperature difference, and the air pressure difference goes beyond a normal range; if at least one of the first temperature difference, the second temperature difference, and the air pressure difference goes beyond the normal range, executing step 1440, i.e., sending a restart instruction to the compressor 320; if none of the at least one of the first temperature difference, the second temperature difference, and the air pressure difference goes beyond the normal range, returning to step 1420.

Step 1450: executing second determination, wherein it is determined whether the number of times which the compressor is restarted within a preset period of time reaches N, and if the number of times which the compressor is restarted within the preset period of time reaches N, ending the process and stopping the first determination, or if the number of times which the compressor is restarted within the preset period of time does not reach N, returning to step 1430 and executing the first determination. N is greater than 1. The preset period of time may be, for example, 1 hour, and by restarting the compressor 320 multiple times within a short period of time, the compressor 320 is ensured to be operated properly as far as possible, so that the magnet is protected from damage, and in addition, the number of times of restarting within the period of time is restricted, so as to prevent an excessively large number of times of restarting.

Typically, the compressor does not report the abnormal state of the compressor, resulting in that it is difficult to detect a failure or an anomaly of the compressor during transport. In this way, it is possible to detect an anomaly of the compressor and perform an effective countermeasure operation in time. By restarting or resetting the protection state of the compressor, the liquid helium in the magnet is protected to the maximum extent, or the magnet is protected from damage.

FIG. 15 illustrates a flow chart of a magnetic resonance system transport method according to some other embodiments of the present application, including the following steps.

Step 1510: sending device operating data to a remote control platform via a remote communication module 913. Step 1520: operating a magnetic resonance system transport apparatus according to an embodiment of the present application in response to an operating instruction from the remote control platform. For example, the remote control platform may display models of the first water-cooling module 620 and the second water-cooling module 630 and display parameter values of components in the models in real time, and when any parameter value is abnormal, an operator may enter a corresponding operating instruction at the remote control platform to perform remote maintenance intervention, or communicate with a transport engineer in real time.

The order of the above steps is not limited in the embodiments of the present application, and the steps may be executed according to a cycle or a temporal order of arrival time depending on a specific circumstance on site, or a step may be started immediately on the basis of an ad hoc event. In addition, the steps may be executed independently, i.e., the implementation of each step is not affected by a result or event in another step.

In addition to any previously indicated modifications, many other variations and replacement arrangements may be devised by those skilled in the art without departing from the substance and scope of the present description, and the appended claims are intended to encompass such modifications and arrangements. Therefore, although the information has been described above in specifics and detailed terms in connection with what is currently considered to be the most practical and preferred aspects, it will be apparent to those of ordinary skill in the art that many modifications can be made, including but not limited to the form, function, mode of operation, and use, without departing from the principles and concepts set forth herein. Likewise, as used herein, in all respects, the examples and embodiments are intended to be illustrative only and should not be construed as limiting in any way.

The purpose of providing the above specific embodiments is to facilitate understanding of the content disclosed in the present invention more thoroughly and comprehensively, but the present invention is not limited to these specific embodiments. Those skilled in the art should understand that various modifications, equivalent replacements, and changes can also be made to the present invention and should be included in the scope of protection of the present invention as long as these changes do not depart from the spirit of the present invention.

1. A magnetic resonance system transport apparatus, comprising an integrated container body, the interior of which being provided with:
a compressor, connected via a cooling pipe to a cold head of a magnetic resonance system arranged outside the integrated container body, so as to supply cooling capacity to the cold head; and
a cooling system, used to perform heat exchange with the compressor in the integrated container body, so as to perform refrigeration on the compressor.

2. The magnetic resonance system transport apparatus according to claim 1, further comprising:
a container, used to accommodate the magnetic resonance system and the integrated container body, the integrated container body being removable from the container.

3. The magnetic resonance system transport apparatus according to claim 2, wherein the container is provided with an opening, via which at least part of the integrated container body is capable of being provided in the container, and via which the integrated container body is in communication with the outside of the container.

4. The magnetic resonance system transport apparatus according to claim 4, wherein the integrated container body comprises an outer side portion in communication with the outside of the container via the opening, and the outer side portion has a shape and a size matching the opening.

5. The magnetic resonance system transport apparatus according to claim 1, wherein the cooling system comprises a water-cooling system.

6. The magnetic resonance system transport apparatus according to claim 5, wherein an air-cooling module is further provided in the integrated container body, and the air-cooling module performs heat exchange with the water-cooling system to cool the cold head.

7. The magnetic resonance system transport apparatus according to claim 6, wherein the air-cooling module delivers cooling air to the cold head via an air duct extending outside the integrated container body.

8. The magnetic resonance system transport apparatus according to claim 5, wherein the cooling system comprises a first water-cooling module and a second water-cooling module, and the first water-cooling module and the second water-cooling module alternately perform heat exchange with the compressor.

9. The magnetic resonance system transport apparatus according to claim 6, wherein the air-cooling module further performs heat exchange with the water-cooling system, so as to output cooling air to the compressor in the integrated container body.

10. The magnetic resonance system transport apparatus according to claim 9, wherein the cooling system comprises a first water-cooling module and a second water-cooling module, and the first water-cooling module and the second water-cooling module alternately supply cooling capacity to the air-cooling module.

11. The magnetic resonance system transport apparatus according to claim 7, wherein a control and communication system is further provided in the integrated container body, the control and communication system comprises a central controller, and the central controller is communicatively coupled to the first water-cooling module and the second water-cooling module, so as to control the first water-cooling module and the second water-cooling module to alternately perform heat exchange with the compressor.

12. The magnetic resonance system transport apparatus according to claim 11, wherein the control and communication system further comprises at least one of the following modules:
a magnet monitoring module, used to receive a main magnet parameter from the magnetic resonance system and send the received main magnet parameter to the central controller;
a human-machine interaction module, controlled by the central controller, so as to display interaction information; and
a remote communication module, used to provide a wireless connection between the central controller and a remote device.

13. The magnetic resonance system transport apparatus according to claim 12, wherein a power supply module is further provided in the integrated container body, and the power supply module is used to receive an external power supply and distribute power to at least one of the compressor, the cooling system, the air-cooling module, and the control and communication system.

14. The magnetic resonance system transport apparatus according to claim 13, wherein the control and communication system further comprises a power supply detection module, used to perform power detection on the power supply module and upload a power detection result to the central controller.

15. The magnetic resonance system transport apparatus according to claim 1, further comprising a shock absorbing and damping apparatus, the shock absorbing and damping apparatus being provided at the bottom of the compressor, the shock absorbing and damping apparatus comprising a base support and a damping part, the damping part comprising an upper portion and a lower portion, the lower portion forming a unitary structure; the damping part further comprising a surface fitting with an upper surface of the base support and two lateral extension portions below the surface, the two lateral extension portions being respectively arranged in a floating mode on two opposite sides of the base support, a space being formed between the upper portion and the lower portion of the damping part, and steel wire rope dampers being respectively mounted between the upper portion of the damping part and the two lateral extension portions the.

16. The magnetic resonance system transport apparatus according to claim 15, wherein a damping space is formed in a horizontal direction between each of the steel wire rope dampers and the lower portion of the damping part.

17. The magnetic resonance system transport apparatus according to claim 1, wherein the integrated container body has a non-rectangular polygonal structure.

18. A magnetic resonance system transport method, wherein a magnetic resonance system is transported by using the magnetic resonance system transport apparatus according to claim 8, and the method comprises executing at least one of the following steps:
periodically switching the first water-cooling module and the second water-cooling module; and
periodically resetting the compressor.

19. The magnetic resonance system transport method according to claim 18, wherein the first water-cooling module comprises a first cooling unit and a first water-cooling circuit that exchanges heat with the first cooling unit, the second water-cooling module comprises a second cooling unit and a second water-cooling circuit that exchanges heat with the second cooling unit, and the method further comprises:
obtaining at least one of a first temperature difference, a second temperature difference, and an air pressure difference in real time, wherein the first temperature difference is a temperature difference between a water outlet temperature and a water inlet temperature of the first cooling unit or the second cooling unit currently in an operating state, the second temperature difference is a temperature difference between a water inlet temperature and a water outlet temperature of the compressor, and the air pressure difference is an air pressure difference between an air-inlet air pressure and a water-outlet air pressure of the compressor;
executing first determination, wherein it is determined whether at least one of the first temperature difference, the second temperature difference, and the air pressure difference goes beyond a normal range; if at least one of the first temperature difference, the second temperature difference, and the air pressure difference goes beyond the normal range, sending a restart instruction to the compressor; if none of the at least one of the first temperature difference, the second temperature difference, and the air pressure difference goes beyond the normal range, returning to the first determination; and
executing second determination, wherein it is determined whether the number of times which the compressor is restarted within a preset period of time reaches N, and if the number of times which the compressor is restarted within the preset period of time reaches N, stopping the first determination, wherein N is greater than 1.
